(19) United States
(12) Patent Application Publication
(10) Pub. No.: US 2006/0229529 A1
(43) Pub. Date: Oct. 12, 2006

(54) System, apparatus, and method for marking orientation of a tissue sample

(76) Inventor: Robert C. Wright, Puyallup, WA (US)

Correspondence Address: BROMBERG & SUNSTEIN LLP, 125 SUMMER STREET, BOSTON, MA, 02110-1618, US

(21) Appl. No.: 11/393,929

(22) Filed: Mar. 30, 2006

(51) Int. Cl. A61B 10/00

(52) U.S. Cl. 600/567

(57) ABSTRACT

Tissue samples are oriented without the use of sutures, specifically using pins that can be inserted through the tissue sample or clips that can be clipped onto the outside surfaces of the tissue sample. The pins and clips are marked, either directly or indirectly, with distinct orientation direction markings such as words, symbols, or colors so that, when the pins or clips are properly inserted or attached, as the case may be, the orientation direction markings indicate the orientation of the tissue sample relative to the body from which it was removed. The orientation direction markings are typically in a form that can be interpreted at least visually, although the orientation direction markings can additionally or alternatively include tactile features. Furthermore, the orientation direction markings can be in a form that show up in x-ray or other examinations of the tissue sample.

### FIELD OF THE INVENTION

The present invention relates generally to orientation of a tissue sample such as a lumpectomy or quadrantectomy, for example, to prepare the tissue sample for radiology or pathology.

### BACKGROUND OF THE INVENTION

In the past, biopsies were generally done open. With modern techniques, an increasing number of biopsies are done with needles, leaving the absolute size of the tumor and its position within the body somewhat in question. More and more, definitive tissue sample removals (e.g., lumpectomies and quadrantectomies) are undertaken without knowing the size of the tumor, and many of these tumors are irregular in shape with protrusions coming out from the central body and/or involvement of the ducts in the surrounding tissue.

From a treatment standpoint, the tumor generally needs to be removed with a margin of normal tissue surrounding it in order to be confident of adequate control of the underlying condition. For example, in the case of breast cancer, the cancer generally needs to be removed with a margin of normal breast tissue surrounding it in order to be confident of adequate control of the cancer within the breast; if the breast is preserved, then the surgical treatment is usually followed by radiation treatments, and any “positive margin” would be a counter-indication to radiation therapy.

For a biopsy patient, the day of surgery might proceed as follows. First, the patient goes to x-ray where a wire localization is performed. The patient then goes to nuclear medicine for injection of radio contrast, and probably imaging an hour or two later, e.g., to detect the location of sentinel lymph nodes in the case of breast cancer. The patient then goes to the surgical suite area where the patient is injected again with blue contrast dye that is used in the sentinel node process as well. The patient then goes to the operating room where the surgeon will usually perform the sentinel node biopsy first and then proceed with the lumpectomy (to reduce the risk of spreading cancer cells from the lumpectomy site to the axilla).

The lumpectomy is done using the needle localization wire to make an educated guess as to the location of the mass, and an educated guess as to what type of margins are needed around the mass. While the patient is still in the operating room the specimen is then sent to radiology, e.g., for specimen mammogram. The radiologist calls the surgeon in the operating room and confirms that the lesion in question has indeed been removed. If there is a concern about margin, the radiologist can communicate with the surgeon at that point that only part of the lesion has been removed and recommend wider margins.

The radiology department then sends the specimen on to the pathology department where the specimen is sliced and put on slides for examination. The pathologist can communicate with the surgeon if there is cancer (either invasive of DCIS) at the resection margins.

If the radiologist and pathologist can only tell the surgeon that the lesion is near a margin without being able to specify the particular margin, then the surgeon then is dealing with lack of orientation. One mechanism for orientation is the location of the wire in relation to the lesion being removed, and the surgeon can make an educated guess as to which side of the tissue sample has a positive margin, but that is truly a guess because the specimen can be rotated 180° around a wire and not provide adequate orientation to be helpful. If there is no good orientation, then the surgeon generally has to remove a centimeter or two of tissue from the biopsy cavity in all directions, which, in the case of breast cancer, can be disfiguring or push the patient into a category needing mastectomy.

To get around this problem, many surgeons have begun marking the orientation of the tissue sample by placing sutures at specific points about the tissue sample. For example, a surgeon might use different numbers of stitches, different types of stitches, and/or a different stitch materials or colors to indicate the various directions of orientation. After orienting the tissue sample, the tissue sample is typically stained in various places (e.g., different colors of ink placed on the various surfaces of the tissue sample). The stained tissue sample can then be processed by a pathologist, who may, for example, x-ray the tissue sample or slice the tissue sample for examination under a microscope. With an oriented specimen, if a margin is positive, a surgeon can go back in and simply remove more tissue in the direction of the positive margin to assure a wider margin, thus increasing the likelihood of success.

The use of sutures does a reasonably good job of orienting the tissue sample. However, the use of sutures requires an inordinate amount of operating room time, which can increase costs (e.g., operating room time, personnel time, etc.) and increase risk to the patient (e.g., the patient remains under anesthesia longer). Furthermore, the use of sutures can increase the likelihood of errors, because the tissue sample is frequently only marked on margins that are somewhat suspicious and because different doctors typically use their own personal suturing schemes for orienting tissue samples (i.e., there is no standardization, which could lead to confusion on the part of nurses and pathologists and ultimately to incorrect specification of the direction of the positive margin). For example, the surgeon will frequently orient the specimen saying “near nipple” or “toward axilla” or “toward clavicle” or “toward naval”, which, although true, does not put the orientation in standard terminology that is appreciated by the pathologist. Then, the orientation terms may be dictated to a nurse, who writes down the orientation terms and passes them along to the pathologist. Because the pathologist must interpret the orientation terms and will use those terms in reporting the lesion back to the surgeon, there is some potential for the specimen to be malrotated (perhaps by 30° or more) based on terminology discrepancies.

### SUMMARY OF THE INVENTION

In accordance with one aspect of the invention there is provided a method for preparing a tissue sample. The method involves removing the tissue sample from a body and orienting the tissue sample by at least one of (a) inserting at least one member through the tissue sample to indicate at least one direction of orientation of the tissue sample and (b) clipping at least one member onto the tissue sample to indicate at least one direction of orientation of the tissue sample, whereby such orienting is accomplished without the use of sutures.

In accordance with another aspect of the invention there is provided apparatus for preparing a tissue sample. The apparatus includes a member for at least one of insertion through the tissue sample and clipping onto the tissue sample and also includes at least one orientation direction marking disposed directly or indirectly on the member for indicating a direction of orientation of the tissue sample, whereby the member and the at least one orientation direction marking indicate a direction of orientation of the tissue sample without the use of sutures.

In accordance with yet another aspect of the invention there is provided a kit for preparing a tissue sample. The kit includes a plurality of members for at least one of insertion through the tissue sample and clipping onto the tissue sample, wherein each member includes a distinct orientation direction marking for indicating a different direction of orientation.

In various embodiments of the invention, the members can include pins for insertion through the tissue sample or clips for clipping onto the tissue sample.

In various alternative embodiments of the present invention, the orientation direction markings can include such things as words, initials, symbols, or colors. The orientation direction markings can be integral to the members or can be included on caps that are coupled to the members. The orientation direction markings may be visible under x-ray inspection of the tissue sample.

In certain embodiments, a plurality of members are used to orient the tissue sample. For example, three pins may be inserted through the tissue sample to indicate the anterior-posterior orientation direction, the superior-inferior orientation direction, and the medial-lateral orientation direction, respectively. Similarly, three or six clips may be clipped onto the tissue sample to indicate the anterior-posterior orientation direction, the superior-inferior orientation direction, and the medial-lateral orientation direction, respectively.

Where pins are used to orient the tissue sample, end caps may be provided to cover the protruding ends of the pins after insertion through the tissue sample.

### BRIEF DESCRIPTION OF THE DRAWINGS

The foregoing and advantages of the invention will be appreciated more fully from the following further description thereof with reference to the accompanying drawings wherein:

FIG. 1 shows a representation of a tissue sample;

FIG. 2 shows an exemplary pin in accordance with an exemplary embodiment of the present invention;

FIG. 3 shows a first set of pins including distinct wording to indicate the three orientation directions;

FIG. 4 shows a second set of pins including distinct initials to indicate the three orientation directions;

FIG. 5 shows a third set of pins including distinct colors to indicate the three orientation directions;

FIG. 6 shows a fourth set of pins including distinct symbols to indicate the three orientation directions;

FIG. 7 shows an exemplary pin assembly including a pin with a pre-installed cap including orientation direction marking(s);

FIG. 8 shows a first set of pin assemblies including distinct wording to indicate the three orientation directions;

FIG. 9 shows a second set of pin assemblies including distinct initials to indicate the three orientation directions;

FIG. 10 shows a third set of pin assemblies including distinct colors to indicate the three orientation directions;

FIG. 11 shows a fourth set of pin assemblies including distinct symbols to indicate the three orientation directions;

FIG. 12 shows an exemplary blank end cap in accordance with an exemplary embodiment of the present invention;

FIG. 13 shows an exemplary tissue sample orientation kit including three pin assemblies and three blank end caps;

FIG. 14 shows a representation of the tissue sample with three pins inserted to indicate the tissue sample orientation;

FIG. 15 shows an exemplary alligator-type clip in accordance with an exemplary embodiment of the present invention;

FIG. 16 shows an exemplary clip assembly in accordance with an exemplary embodiment of the present invention;

FIG. 17 shows a representation of the tissue sample with six clips attached to indicate the tissue sample orientation; and

FIG. 18 shows an alternate representation of a tissue sample with three pins inserted to indicate the tissue sample orientation.

### DETAILED DESCRIPTION OF SPECIFIC EMBODIMENTS

In exemplary embodiments of the present invention, tissue samples are oriented without the use of sutures, specifically using pins that can be inserted through the tissue sample or clips that can be clipped onto the outside surfaces of the tissue sample. The pins and clips can be applied quickly and accurately by the surgeon and provide a standardized orientation scheme so as to reduce the chances of errors from different orientation schemes and unreliable communication of orientation terms between the surgeon, nurse, and pathologist.

The pins and clips are marked, either directly or indirectly, with distinct orientation direction markings such as words, symbols, or colors so that, when the pins or clips are properly inserted or attached, as the case may be, the orientation direction markings indicate the orientation of the tissue sample relative to the body from which it was removed. The orientation direction markings are typically in a form that can be interpreted at least visually, although the orientation direction markings can additionally or alternatively include tactile features. Furthermore, the orientation direction markings can be in a form that show up in x-ray or other examinations of the tissue sample.

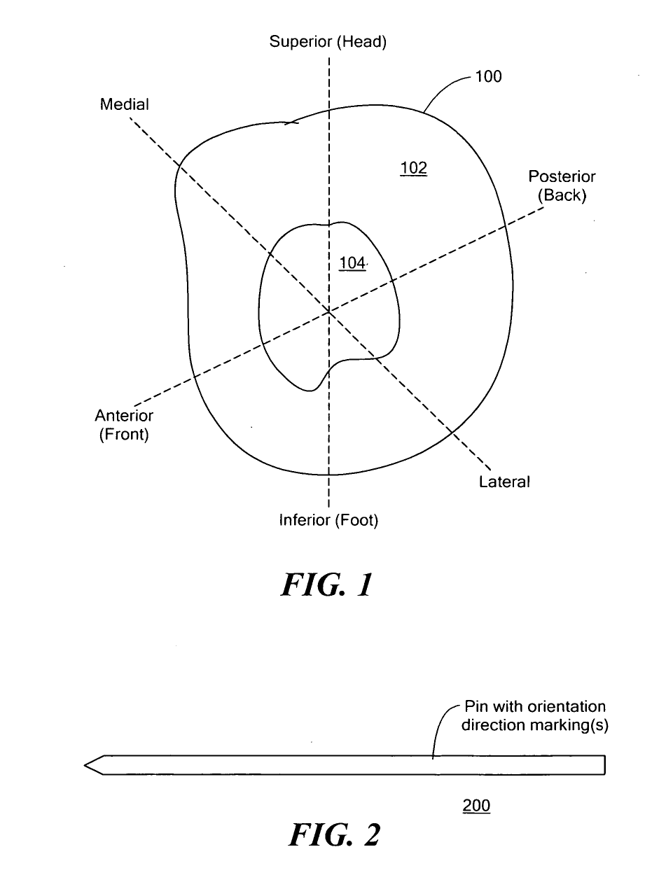

FIG. 1 shows a representation of a tissue sample 100. The tissue sample 100 includes a tumor 104 with some amount of surrounding tissue 102. The orientation of the tissue sample 100 relative to the body from which it is removed is indicated by dotted lines along three axes, specifically the medial/lateral axis, the anterior/posterior axis, and the superior/inferior axis.

In certain exemplary embodiments of the present invention, one or more pins are inserted through the tissue sample in order to orient the tissue sample. FIG. 2 shows an exemplary pin 200 in accordance with an exemplary embodiment of the present invention. The pin 200 can be, for example, a solid or hollow pin of stainless steel (e.g., 0.050 stainless steel) or other material that is sharpened at one end (e.g, with a trocar point) and marked at one or both ends with orientation direction marking(s). Generally, a plurality of pins would be used to orient a tissue sample. For example, three pins would generally be used to indicate the three orientation axes shown in FIG. 1.

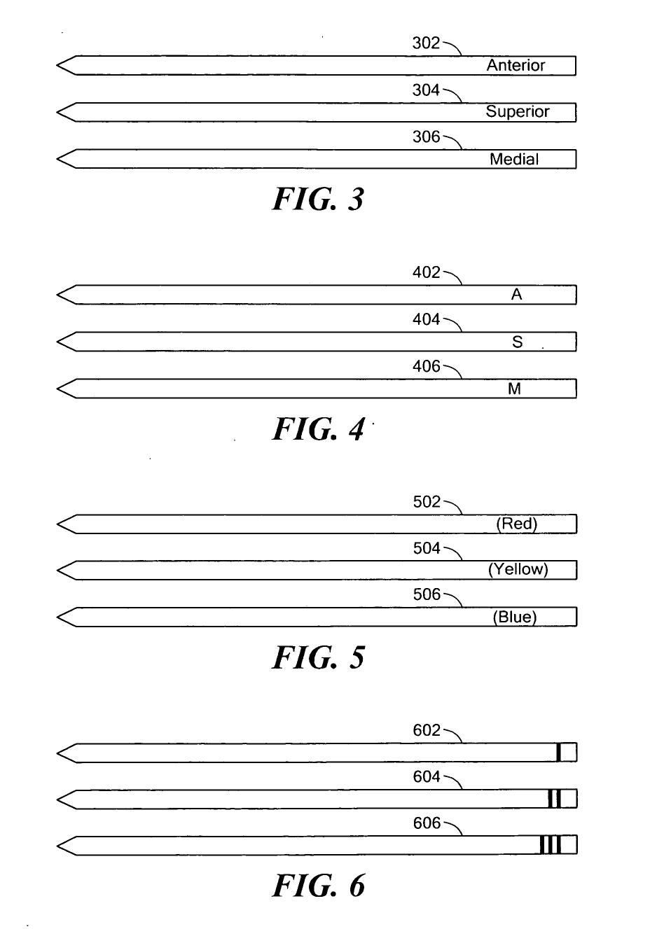

FIG. 3 shows a first set of pins including distinct wording to indicate the three orientation directions. Specifically, a first pin 302 marked with the word “Anterior” would generally be used to indicate the anterior/posterior axis (e.g., by inserting the pin at the anterior point of the tissue sample so that the word “Anterior” protrudes from the anterior point of the tissue sample and the other end protrudes from the posterior point of the tissue sample), a second pin 304 marked with the word “Superior” would generally be used to indicate the superior/inferior axis (e.g., by inserting the pin at the superior point of the tissue sample so that the word “Superior” protrudes from the superior point of the tissue sample and the other end protrudes from the inferior point of the tissue sample), and a third pin 306 marked with the word “Medial” would generally be used to indicate the medial/lateral axis (e.g., by inserting the pin at the medial point of the tissue sample so that the word “Medial” protrudes from the medial point of the tissue sample and the other end protrudes from the lateral point of the tissue sample). The wording can be printed, painted, etched, molded, or otherwise formed in or on the pins.

FIG. 4 shows a second set of pins including distinct initials to indicate the three orientation directions. Specifically, a first pin 402 marked with the initial “A” would generally be used to indicate the anterior/posterior axis (e.g., by inserting the pin at the anterior point of the tissue sample so that the initial “A” protrudes from the anterior point of the tissue sample and the other end protrudes from the posterior point of the tissue sample), a second pin 404 marked with the initial “S” would generally be used to indicate the superior/inferior axis (e.g., by inserting the pin at the superior point of the tissue sample so that the initial “S” protrudes from the superior point of the tissue sample and the other end protrudes from the inferior point of the tissue sample), and a third pin 406 marked with the initial “M” would generally be used to indicate the medial/lateral axis (e.g., by inserting the pin at the medial point of the tissue sample so that the initial “M” protrudes from the medial point of the tissue sample and the other end protrudes from the lateral point of the tissue sample). The initials can be printed, painted, etched, molded, or otherwise formed in or on the pins.

FIG. 5 shows a third set of pins including distinct colors to indicate the three orientation directions. Specifically, a first pin 502 marked with the color red might be used to indicate the anterior/posterior axis (e.g., by inserting the pin at the anterior point of the tissue sample so that the red end protrudes from the anterior point of the tissue sample and the other end protrudes from the posterior point of the tissue sample), a second pin 504 marked with the color yellow might be used to indicate the superior/inferior axis (e.g., by inserting the pin at the superior point of the tissue sample so that the yellow end protrudes from the superior point of the tissue sample and the other end protrudes from the inferior point of the tissue sample), and a third pin 506 marked with the color blue might be used to indicate the medial/lateral axis (e.g., by inserting the pin at the medial point of the tissue sample so that the blue end protrudes from the medial point of the tissue sample and the other end protrudes from the lateral point of the tissue sample). The colors can be printed, painted, otherwise formed in or on the pins.

FIG. 6 shows a fourth set of pins including distinct symbols to indicate the three orientation directions. Specifically, a first pin 602 marked with a single ring might be used to indicate the anterior/posterior axis (e.g., by inserting the pin at the anterior point of the tissue sample so that the end with the single ring protrudes from the anterior point of the tissue sample and the other end protrudes from the posterior point of the tissue sample), a second pin 604 marked with two rings might be used to indicate the superior/inferior axis (e.g., by inserting the pin at the superior point of the tissue sample so that the end with two rings protrudes from the superior point of the tissue sample and the other end protrudes from the inferior point of the tissue sample), and a third pin 606 marked with three rings might be used to indicate the medial/lateral axis (e.g., by inserting the pin at the medial point of the tissue sample so that the end with three rings protrudes from the medial point of the tissue sample and the other end protrudes from the lateral point of the tissue sample). The rings can be printed, painted, etched, molded, or otherwise formed in or on the pins.

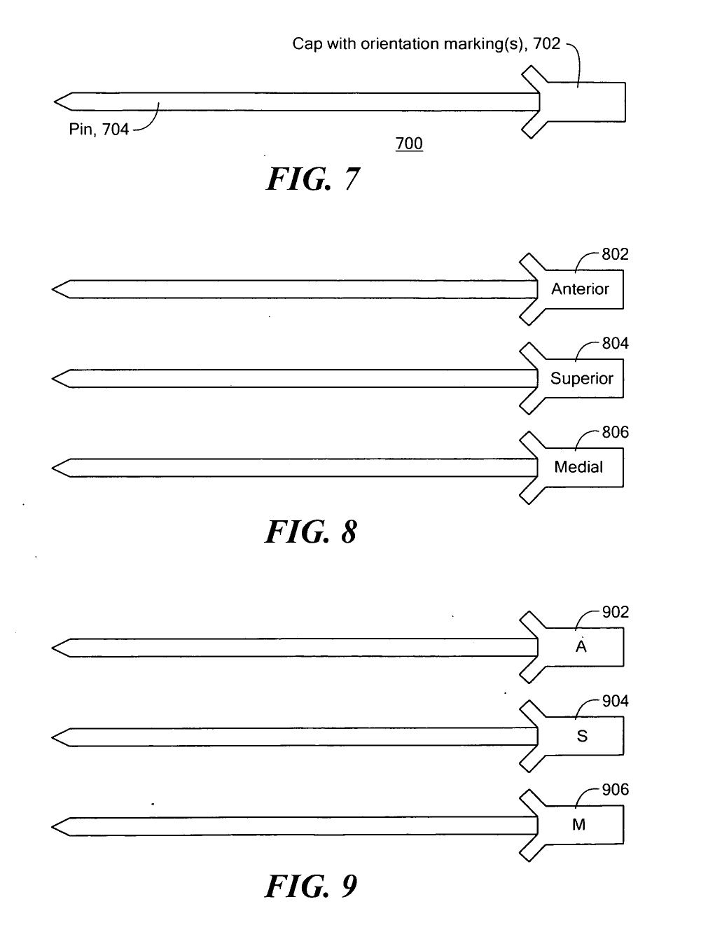

In order to facilitate handling and insertion of the pins, the pins can be provided or otherwise fitted with caps that include the orientation direction marking(s). FIG. 7 shows an exemplary pin assembly 700 including a pin 704 with a pre-installed cap 702 including orientation direction marking(s). The caps can be installed by the manufacturer/seller or by one of the medical staff (e.g., the surgeon or nurse). The caps can include a flattened surface to prevent the pin assemblies from rolling when placed on a table. As above, the pin 704 can be, for example, a solid or hollow pin of stainless steel or other material that is sharpened at the opposite end from the cap. Generally, a plurality of pin assemblies would be used to orient a tissue sample. For example, three pin assemblies would generally be used to indicate the three orientation axes shown in FIG. 1.

FIG. 8 shows a first set of pin assemblies including distinct wording to indicate the three orientation directions. Specifically, a first pin assembly 802 marked with the word “Anterior” would generally be used to indicate the anterior/posterior axis (e.g., by inserting the pin at the anterior point of the tissue sample so that the cap with the word “Anterior” protrudes from the anterior point of the tissue sample and the other end protrudes from the posterior point of the tissue sample), a second pin assembly 804 marked with the word “Superior” would generally be used to indicate the superior/inferior axis (e.g., by inserting the pin at the superior point of the tissue sample so that the cap with the word “Superior” protrudes from the superior point of the tissue sample and the other end protrudes from the inferior point of the tissue sample), and a third pin assembly 806 marked with the word “Medial” would generally be used to indicate the medial/lateral axis (e.g., by inserting the pin at the medial point of the tissue sample so that the cap with the word “Medial” protrudes from the medial point of the tissue sample and the other end protrudes from the lateral point of the tissue sample). The wording can be printed, painted, etched, molded, or otherwise formed in or on the caps.

FIG. 9 shows a second set of pin assemblies including distinct initials to indicate the three orientation directions. Specifically, a first pin assembly 902 marked with the initial “A” would generally be used to indicate the anterior/posterior axis (e.g., by inserting the pin at the anterior point of the tissue sample so that the cap with the initial “A” protrudes from the anterior point of the tissue sample and the other end protrudes from the posterior point of the tissue sample), a second pin assembly 904 marked with the initial “S” would generally be used to indicate the superior/inferior axis (e.g., by inserting the pin at the superior point of the tissue sample so that the cap with the initial “S” protrudes from the superior point of the tissue sample and the other end protrudes from the inferior point of the tissue sample), and a third pin assembly 906 marked with the initial “M” would generally be used to indicate the medial/lateral axis (e.g., by inserting the pin at the medial point of the tissue sample so that the cap with the initial “M” protrudes from the medial point of the tissue sample and the other end protrudes from the lateral point of the tissue sample). The initials can be printed, painted, etched, molded, or otherwise formed in or on the caps.

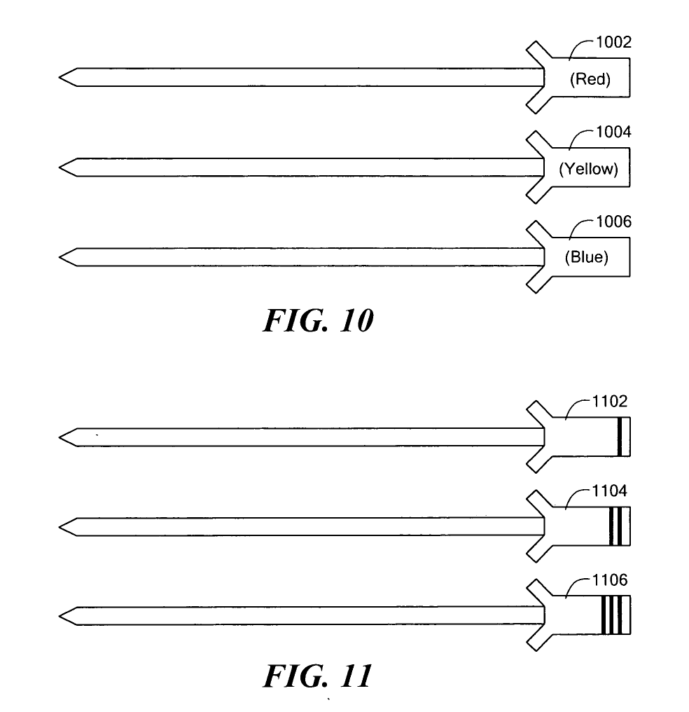

FIG. 10 shows a third set of pin assemblies including distinct colors to indicate the three orientation directions. Specifically, a first pin assembly 1002 marked with the color red might be used to indicate the anterior/posterior axis (e.g., by inserting the pin at the anterior point of the tissue sample so that the red cap protrudes from the anterior point of the tissue sample and the other end protrudes from the posterior point of the tissue sample), a second pin assembly 1004 marked with the color yellow might be used to indicate the superior/inferior axis (e.g., by inserting the pin at the superior point of the tissue sample so that the yellow cap protrudes from the superior point of the tissue sample and the other end protrudes from the inferior point of the tissue sample), and a third pin assembly 1006 marked with the color blue might be used to indicate the medial/lateral axis (e.g., by inserting the pin at the medial point of the tissue sample so that the blue cap protrudes from the medial point of the tissue sample and the other end protrudes from the lateral point of the tissue sample). The colors can be printed, painted, otherwise formed in or on the caps.

FIG. 11 shows a fourth set of pin assemblies including distinct symbols to indicate the three orientation directions. Specifically, a first pin assembly 1102 marked with a single ring might be used to indicate the anterior/posterior axis (e.g., by inserting the pin at the anterior point of the tissue sample so that the cap with the single ring protrudes from the anterior point of the tissue sample and the other end protrudes from the posterior point of the tissue sample), a second pin assembly 1104 marked with two rings might be used to indicate the superior/inferior axis (e.g., by inserting the pin at the superior point of the tissue sample so that the cap with two rings protrudes from the superior point of the tissue sample and the other end protrudes from the inferior point of the tissue sample), and a third pin assembly 1106 marked with three rings might be used to indicate the medial/lateral axis (e.g., by inserting the pin at the medial point of the tissue sample so that the cap with three rings protrudes from the medial point of the tissue sample and the other end protrudes from the lateral point of the tissue sample). The rings can be printed, painted, etched, molded, or otherwise formed in or on the caps.

It should be noted that the embodiments described above are exemplary only. The present invention is not limited to any particular type of pin, type of cap, type of orientation direction marking, or form of orientation direction marking. It will be appreciated that different wording could be used to indicate a particular orientation axis (e.g., “front” instead of “anterior”).

The embodiments described above would be sufficient to orient a tissue sample. In practice, though, it would not be prudent for anyone to handle the tissue sample with the sharpened ends of the pins protruding from the tissue sample. Therefore, alternative embodiments of the present invention may provide end caps to be placed on the sharpened ends of the pins after insertion through the tissue sample. These end caps can be virtually identical to the pre-installed end caps in form, but either blank (i.e., without any orientation direction markings) or with appropriate orientation direction markings so as to avoid confusion (e.g., an end cap might be marked “Posterior” for use with the pin or pin assembly marked “Anterior”).

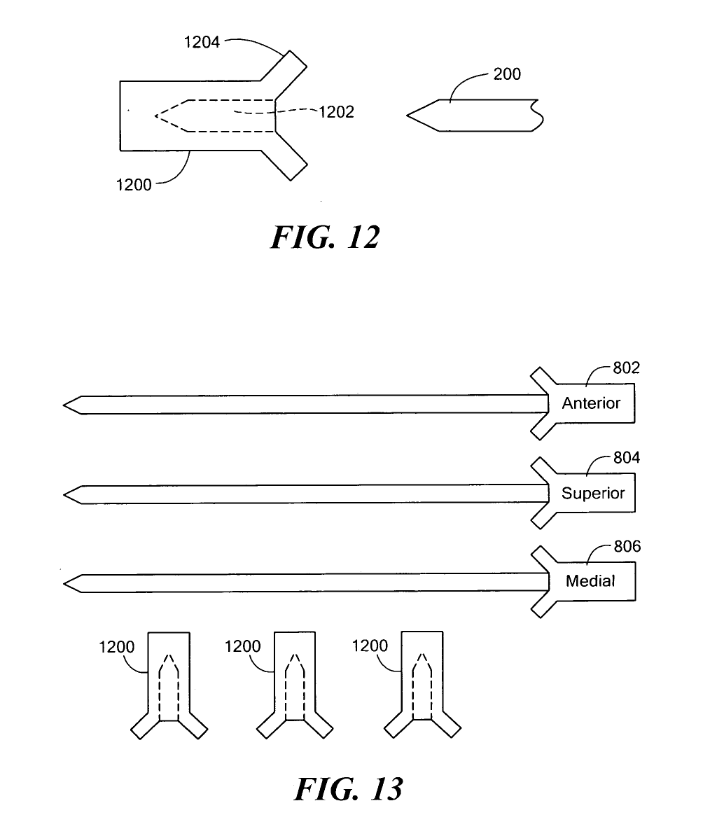

FIG. 12 shows an exemplary blank end cap 1200 in accordance with an exemplary embodiment of the present invention. The end cap typically includes an interior cavity 1202 configured to receive the sharpened end of a pin 200. The end cap may also include a flange 1204 to help guide the pin 200 into the cavity 1202 while also helping to protect a person's finger from being accidentally pierced by the pin 200.

Thus, a kit for tissue sample orientation might include three pins or pin assemblies (marked respectively for the three orientation directions) and three end caps. FIG. 13 shows an exemplary tissue sample orientation kit 1300 including three pin assemblies 802, 804, and 806 and three blank end caps 1200. The components of the kit 1300 would generally be provided sterilized.

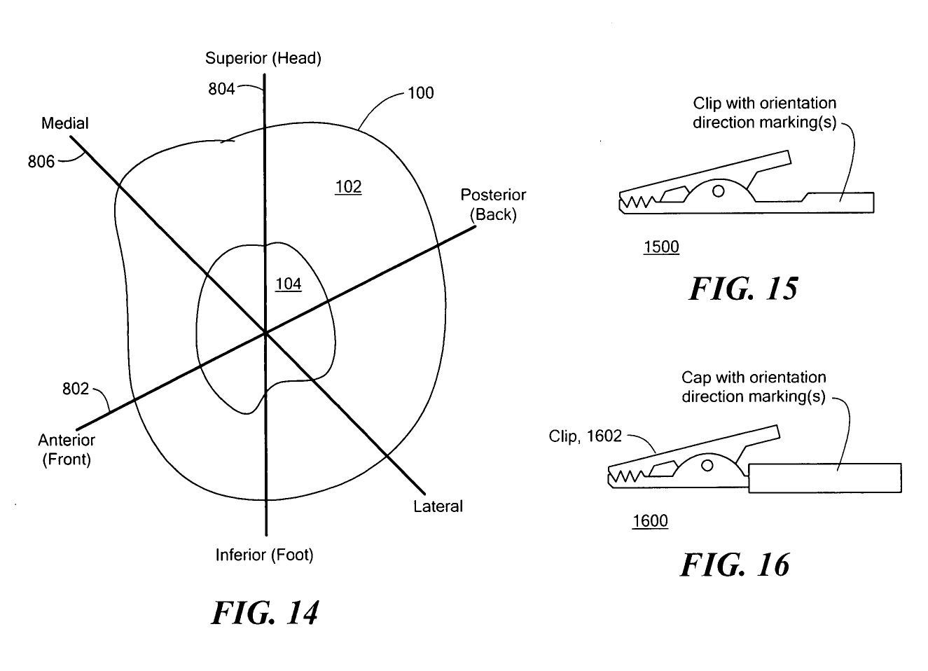

FIG. 14 shows a representation of the tissue sample 100 with three pins inserted to indicate the tissue sample orientation. Specifically, pin assembly 802 is inserted from anterior point to posterior point, pin assembly 804 is inserted from superior point to inferior point, and pin assembly 806 is inserted from medial point to lateral point.

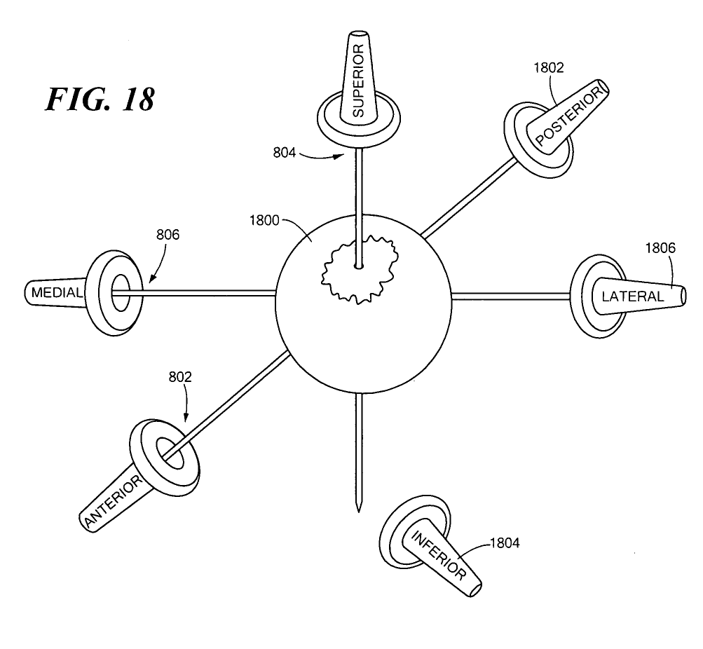

FIG. 18 shows an alternate representation of a tissue sample with three pins inserted to indicate the tissue sample orientation. Specifically, pin assemblies 802, 804, and 806 are inserted through the tissue sample 1800 to indicate the three axes of orientation. Three end caps 1802, 1804, and 1806 are provided to respectively cover the protruding ends of the pin assemblies 802, 804, and 806. In this example, the end caps are not blank, but rather include complementary wording to the pre-installed caps of the cap assemblies. Specifically, end cap 1802 is labeled “Posterior” for use with the pin assembly labeled “Anterior,” end cap 1804 is labeled “Inferior” for use with the pin assembly labeled “Superior,” and end cap 1806 is labeled “Lateral” for use with the pin assembly labeled “Medial.”

In other exemplary embodiments of the present invention, one or more clips are attached to the surface of the tissue sample in order to orient the tissue sample. The clips can be alligator-type clips or other types of clips (e.g., bulldog clips, grabber clips, parrot clips, etc.) that are capable of being attached to the tissue sample. The clips can be formed from stainless steel or other material and marked with orientation direction marking(s). Generally, a plurality of clips would be used to orient a tissue sample. For example, at least three clips (and possibly six clips) would generally be used to indicate the three orientation axes shown in FIG. 1.

FIG. 15 shows an exemplary alligator-type clip 1500 in accordance with an exemplary embodiment of the present invention. The clip 1500 is directly marked with orientation direction marking(s). For example, the orientation direction markings can be in the form of words, initials, symbols, or colors on the handle of the clip as shown and described above with reference to FIGS. 3-6.

FIG. 16 shows an exemplary clip assembly 1600 in accordance with an exemplary embodiment of the present invention. The clip assembly 1600 includes a clip 1602 and a pre-installed cap 1604 with orientation direction marking(s). For example, the orientation direction markings can be in the form of words, initials, symbols, or colors on the cap as shown and described above with reference to FIGS. 8-11.

A kit for tissue sample orientation might include three clips or clip assemblies marked respectively for three orientation points or might include six clips or clip assemblies marked respectively for six orientation points. The components of the kit would generally be provided sterilized.

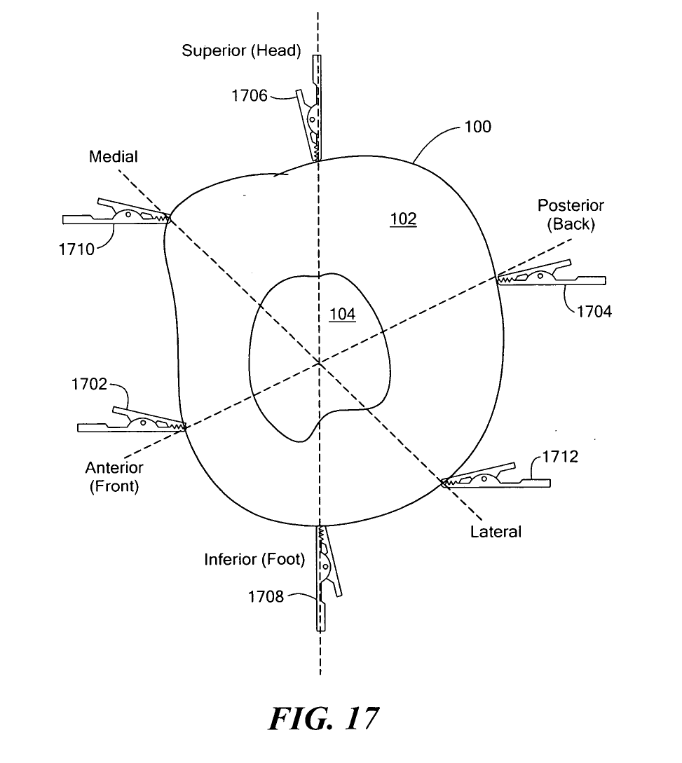

FIG. 17 shows a representation of the tissue sample 100 with six clips attached to indicate the tissue sample orientation. Specifically, clips 1702 and 1704 are respectively attached to the tissue sample at the anterior and posterior points, clips 1706 and 1708 are respectively attached to the tissue sample at the superior and inferior points, and clips 1710 and 1712 are respectively attached to the tissue sample at the medial and lateral points.

As discussed above, the orientation direction markings can be in a form that show up in x-ray or other examinations of the tissue sample. Thus, for example, the orientation direction markings could be printed onto the pins/clips or caps using a metallic ink.

It will be appreciated that combinations of orientation direction markings can be employed. For example, a cap could be color coded and also include appropriate wording printed in metallic ink so that the appropriate device could be quickly identified by color but also identified by the wording (e.g., to accommodate those who are color-blind) and could also be identified in an x-ray picture of the tissue sample.

Thus, the present invention may be embodied as a method, apparatus, and kit for preparing a tissue sample in which one or more members are inserted through the tissue sample and/or clipped onto the tissue sample such that the tissue sample is oriented without the use of sutures.

The present invention may be embodied in other specific forms without departing from the true scope of the invention. The described embodiments are to be considered in all respects only as illustrative and not restrictive.

What is claimed is:

1. A method for preparing a tissue sample, the method comprising:
removing the tissue sample from a body; and
orienting the tissue sample by at least one of (a) inserting at least one member through the tissue sample to indicate at least one direction of orientation of the tissue sample and (b) clipping at least one member onto the tissue sample to indicate at least one direction of orientation of the tissue sample, whereby such orienting is accomplished without the use of sutures.

2. A method according to claim 1, wherein each member includes at least one distinct orientation direction marking.

3. A method according to claim 2, wherein the at least one distinct orientation direction marking includes a word.

4. A method according to claim 2, wherein the at least one distinct orientation direction marking includes an initial.

5. A method according to claim 2, wherein the at least one distinct orientation direction marking includes a symbol.

6. A method according to claim 2, wherein the at least one distinct orientation direction marking includes a color.

7. A method according to claim 2, wherein the at least one orientation direction marking is integral to the at least one member.

8. A method according to claim 2, wherein the at least one orientation direction marking is integral to a cap that is coupled to the at least one member.

9. A method according to claim 2, wherein the at least one orientation direction marking is visible under x-ray inspection of the tissue sample.

10. A method according to claim 1, wherein orienting the tissue sample includes at least one of (a) inserting a plurality of members through the tissue sample and (b) clipping a plurality of members onto the tissue sample.

11. A method according to claim 10, wherein the plurality of members includes at least three members.

12. A method according to claim 11, wherein the plurality of members includes:
a first member for indicating an anterior-posterior orientation direction;
a second member for indicating a superior-inferior orientation direction; and
a third member for indicating a medial-lateral orientation direction.

13. A method according to claim 1, wherein the at least one member includes a pin.

14. A method according to claim 1, wherein the at least one member includes a clip.

15. Apparatus for orienting a tissue sample, the apparatus comprising:
a member for at least one of insertion through the tissue sample and clipping onto the tissue sample; and
at least one orientation direction marking disposed directly or indirectly on the member for indicating a direction of orientation of the tissue sample, whereby the member and the at least one orientation direction marking indicate a direction of orientation of the tissue sample without the use of sutures.

16. Apparatus according to claim 15, wherein the at least one orientation direction marking includes a word.

17. Apparatus according to claim 15, wherein the at least one orientation direction marking includes an initial.

18. Apparatus according to claim 15, wherein the at least one orientation direction marking includes a symbol.

19. Apparatus according to claim 15, wherein the at least one orientation direction marking includes a color.

20. Apparatus according to claim 15, wherein the at least one orientation direction marking is integral to the member.

21. Apparatus according to claim 15, further comprising:
a cap coupled to the member, wherein the at least one orientation direction marking is integral to the cap.

22. Apparatus according to claim 15, wherein the at least one orientation direction marking is visible under x-ray inspection of the tissue sample.

23. Apparatus according to claim 15, wherein the at least one orientation direction marking indicates one of:
an anterior-posterior orientation direction;
a superior-inferior orientation direction; and
a medial-lateral orientation direction.

24. Apparatus according to claim 15, wherein the member is a pin.

25. Apparatus according to claim 15, wherein the member is a clip.

26. A kit for preparing a tissue sample, the kit comprising:
a plurality of members for at least one of insertion through the tissue sample and clipping onto the tissue sample, wherein each member includes a distinct orientation direction marking for indicating a different direction of orientation.

27. A kit according to claim 26, wherein each member includes a cap that includes the distinct orientation direction marking.

28. A kit according to claim 26, wherein the members are pins, and wherein the kit further comprises a plurality of end caps for covering protruding ends of the pins after insertion through the tissue sample.
